(19) United States
(12) Patent Application Publication
(10) Pub. No.: US 2006/0178318 A1
(43) Pub. Date: Aug. 10, 2006

(54) Use of aurora kinase inhibitors for reducing the resistance of cancer cells

(76) Inventors: Shubha Anand, Cambridge (GB); Ashok Venkitaraman, Cambridge (GB)

Correspondence Address: NIXON & VANDERHYE, PC, 901 NORTH GLEBE ROAD, 11TH FLOOR, ARLINGTON, VA, 22203, US

(21) Appl. No.: 10/563,042

(22) PCT Filed: Jul. 3, 2003
(86) PCT No.: PCT/GB03/02862

§ 371 Date: Mar. 13, 2006

(51) Int. Cl. A61K 48/00; A61K 31/7048; A61K 31/5377; A61K 31/337

(52) U.S. Cl. 514/27; 514/44; 514/234.2; 514/449

(57) ABSTRACT

This invention relates to the use of anti-cancer agents that inhibit mitotic spindle assembly in target cells, including taxanes such as paclitaxel, and in particular to methods and means for predicting and/or reducing the resistance of cancer cells to such agents. Over-expression of aurora kinases, such as Aurora A, mediates resistance to such anti-cancer agents and the resistance of a cancer cell may be reduced by inhibiting aurora kinases and/or predicted by measuring the expression or activity of aurora kinases within the cell

This invention relates to the use of anti-cancer agents that inhibit mitotic spindle assembly in target cells ar thereby induce apoptosis, and in particular to methods and means for predicting and/or reducing the resistance of cancer cells to such agents.

Many chemotherapeutic agents inhibit the assembly of the mitotic spindle, for example by targeting mitotic processes such as the kinetochore-microtubule dynamics which are monitored by the spindle assembly checkpoint. These agents include paclitaxel (taxol™) and other taxanes, which are widely used in the treatment of refractory ovarian cancer, breast cancer, and other type of epithelial cancer (Rowinsky, E. K. & Donehower, R. C. (1991) Pharmacol. Ther. 52, 35-84). Paclitaxel binds microtubules and causes kinetic suppression of microtubule dynamics by enhancing microtubule polymerization. This inhibits cell cycle progression, causing cells to arrest at the metaphase-anaphase transition, and subsequently leads to apoptosis (Wang, T. H. et al (2000) Cancer 88, 2619-2628)

Although agents which inhibit mitotic spindle assembly are effective in the treatment of cancer, some tumours appear to be resistant to the apoptotic effects of these agents.

The present inventors have found that AURORA-A over-expression, which is estimated to occur in 12%-62% of breast and colorectal cancers, dysregulates the spindle checkpoint during carcinogenesis and reduces the sensitivity of cells to agents that inhibit mitotic spindle assembly. Inhibiting the activity of Aurora kinase may therefore improve the responsiveness of cancer cells to such agents.

A first aspect of the invention provides the use of an Aurora kinase inhibitor and a mitotic spindle assembly inhibitor in the manufacture of a medicament for use in the treatment of cancer in an individual.

An agent which inhibits mitotic spindle assembly may, for example, bind microtubules and alter microtubule polymerization leading to the inhibition cell cycle progression and eventually to apoptosis. Examples of mitotic spindle assembly inhibiting agents include taxanes, for example paclitaxel and analogues or derivatives thereof.

Taxanes are complex esters consisting of a 15-member taxane ring system linked to a four-member oxetan ring. Preferred taxanes are those having the constituents known in the art to be required for enhancement of microtubule formation, e.g., paclitaxel and docetaxel. The structures of paclitaxel and docetaxel differ in substitutions at the C-10 taxane ring position and on the ester side chain attached at C-13. Docetaxel has t-butoxycarbonyl instead of benzoyl on the amino group of (2R,3S)-phenylisoserine moiety at the C-13 position and a hydroxyl group instead of acetoxy group at C-10. The structures of paclitaxel and docetaxel are well known in the art.

Other taxanes suitable for use as described herein are paclitaxel derivatives having structural variations along the portion of the paclitaxel molecule comprising carbons 6-12, with oxygen functions at C-7, C-9 and C-10. Many such derivatives are known in the art, and it is known that such derivatives exhibit biological activity that is comparable to the bioactivity of paclitaxel. For example, acylation of the C-7 hydroxyl group, or its replacement with hydrogen, does not significantly reduce the activity of paclitaxel. Additionally, replacement of the 10-acetoxy group with hydrogen causes only a small reduction in activity.

Reduction of the C-9 carbonyl group to an α-OH group is known to cause a slight increase in tubulin-assembly activity. Additionally, it is known that a rearrangement product with a cyclopropane ring bridging the seven and eight-position is almost as cytotoxic as paclitaxel. It has also been reported that m-substituted benzoyl derivatives are more active than their p-substituted analogues, and are often more active than paclitaxel itself.

Another paclitaxel analog suitable for use as described herein is A-nor-paclitaxel. This analog has tubulin-assembly activity that is only three times less than that of paclitaxel. A-nor-paclitaxel and paclitaxel have very similar molecular shapes, which may explain their similar tubulin-assembly activities.

Other suitable taxanes are taxasm, 7-epipaclitaxel, t-acetyl paclitaxel, 10-desacetyl-paclitaxel, 10-desacetyl-7-epipaclitaxel, 7-xylosylpaclitaxel, 10-desacetyl-7-glutarylpaclitaxel, 7-N,N-dimethylglycylpaclitaxel, 7-L-alanylpaclitaxel, and mixtures thereof.

Cancers cells suitable for treatment in accordance with the invention may show amplification of one or more Aurora kinase genes (i.e. an increase in copy number of Aurora A, B and/or C relative to non-cancer cells) and/or elevated expression of one or more Aurora kinase proteins relative to non-cancer cells (which may occur, for example in the absence of gene amplification). Preferably, a cancer cell suitable for treatment exhibits spindle checkpoint dysfunction and/or resistance to inhibitors of mitotic spindle assembly. Cancers in which Aurora kinases may be over expressed include epithelial cancers such as skin, thyroid, colon, pancreas, lung, prostate, ovarian, cervical or breast cancer and other cancers such as liver, kidney or brain cancer.

An Aurora kinase inhibitor may inhibit or reduce the activity in a cell of an Aurora kinase, for example one or more of Aurora A, B or C. The amino acid and nucleotide sequences of human Aurora A, B and C are available on the NCBI Entrez database; human Aurora-A protein: O14965, coding sequence: NM_{—}003600, human Aurora-B protein: Q96GD4, coding sequence: NM_{—}004217 and human Aurora-C protein: BAA76292, coding sequence AB017332. In some preferred embodiments, an Aurora kinase inhibitor may inhibit the activity of Aurora A.

Aurora kinase activity may be determined by contacting the kinase polypeptide with the substrate of said polypeptide under conditions in which the kinase normally phosphorylates the substrate. The depletion of unphosphorylated substrate or the formation of phosphorylated substrate by the kinase polypeptide may then be determined. Suitable substrate molecules may include histone H3 or analogues or derivatives thereof.

Phosphorylation of a substrate such as histone H3 may be determined by any convenient method. For example, it may be detected by methods employing radiolabelled ATP and optionally, a scintillant. A radiolabelled protein may be detected by capturing it on a solid substrate using an antibody or other specific binding molecule directed against the protein and immobilised to the substrate, the substrate being impregnated with a scintillant—such as in a standard scintillation proximity assay. Phosphorylation is then determined via measurement of the incorporation of radioactive phosphate.

Alternatively, radiolabelled phosphate incorporation may be determined by precipitation with acid, such as trichloroacetic acid, and collection of the precipitate on a nitrocellulose filter paper, followed by measurement of incorporation of radiolabelled phosphate.

Phosphorylation may also be detected by methods employing an antibody or other specific binding molecule which binds the phosphorylated polypeptide with a different affinity to unphosphorylated polypeptide. Such antibodies may be obtained by means of any standard technique as discussed elsewhere herein. Binding of a specific binding molecule which discriminates between the phosphorylated and non-phosphorylated form of a polypeptide may be assessed using any technique available to those skilled in the art, for example immunoblotting.

In other embodiments, Aurora kinase activity may be determined by a functional assay, for example by detecting the formation of a central spindle during anaphase, for example by immunofluorescence microscopy.

Over expression of Aurora-A in mammalian cells causes centrosome amplification, aneuploidy and multi-nucleation. Aurora-A activity may also be measured by counting centrosome numbers per cell, by determining chromosome number or by counting the percentage of bi-nucleate or multi-nucleate cells using conventional techniques. Expression of Aurora-A or Aurora-B in yeast cells (S. cerevisiae) causes cell death, and this may also be used as a measure of Aurora kinase activity.

The ability of a compound to inhibit Aurora kinase activity may be determined by measuring Aurora kinase activity in the presence and absence of the compound. A reduction in activity in the presence of the compound is indicative that the compound is an inhibitor.

Suitable Aurora kinase inhibitors include compounds such as 4-(4-(N benzoylamino)anilino)-6-methoxy-7-(3-(1 morpholino)propoxy)quinazoline (ZM447439: Ditchfield et al J Cell Biol 161:267-80 (2003) and Hesperadin (Haaf et al J Cell Biol 161:281-94 (2003). Other compounds suitable for use as Aurora kinase inhibitors are described in Vankayalapati H et al Mol Cancer Ther. 2003 2(3): 283-9.

Other suitable Aurora kinase inhibitors may include antibody molecules directed to the active site of an Aurora kinase, for example Aurora A, B or C. Candidate inhibitor antibody molecules may be characterised and their binding regions determined to provide single chain antibodies and fragments thereof that inhibit Aurora kinase activity.

Antibody molecules may be obtained using techniques which are standard in the art. Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and screened, preferably using binding of antibody to antigen of interest. For instance, Western blotting techniques or immunoprecipitation may be used (Armitage et al., 1992, Nature 357: 80-82). Isolation of antibodies and/or antibody-producing cells from an animal may be accompanied by a step of sacrificing the animal.

As an alternative or supplement to immunising a mammal with a peptide, an antibody molecule specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with any of the proteins (or fragments), or may be one constructed using sequences obtained from an organism which has been exposed to the antigen of interest.

Antibody molecules may be modified in a number of ways and the term “antibody molecule” should be construed as covering any binding substance having an immunoglobulin binding domain with the required specificity. Thus the invention covers antibody fragments, derivatives, functional equivalents and homologues of antibodies.

An alternative approach to Aurora kinase inhibition employs regulation at the nucleic acid level to inhibit activity or function by down-regulating expression of Aurora kinases.

For instance, expression of a gene may be inhibited using anti-sense, sense or RNAi technology. The use of these approaches to down-regulate gene expression is now well-established in the art.

Anti-sense oligonucleotides may be designed to hybridise to the complementary sequence of nucleic acid, pre-mRNA or mature mRNA, interfering with the production of MRIP1 polypeptide so that its expression is reduced or completely or substantially completely prevented. In addition to targeting coding sequence, anti-sense techniques may be used to target control sequences of a gene, e.g. in the 5′ flanking sequence, whereby the anti-sense oligonucleotides can interfere with expression control sequences. The construction of anti-sense sequences and their use is described for example in Peyman and Ulman, Chemical Reviews, 90:543-584, (1990) and Crooke, Ann. Rev. Pharmacol. Toxicol. 32:329-376, (1992).

Oligonucleotides may be generated in vitro or ex vivo for administration or anti-sense RNA may be generated in vivo within cells in which down-regulation is desired. Thus, double-stranded DNA may be placed under the control of a promoter in a “reverse orientation” such that transcription of the anti-sense strand of the DNA yields RNA which is complementary to normal mRNA transcribed from the sense strand of the target gene. The complementary anti-sense RNA sequence is thought then to bind with mRNA to form a duplex, inhibiting translation of the endogenous mRNA from the target gene into protein. Whether or not this is the actual mode of action is still uncertain. However, it is established fact that the technique works.

The complete sequence corresponding to the coding sequence in reverse orientation need not be used. For example fragments of sufficient length may be used. It is a routine matter for the person skilled in the art to screen fragments of various sizes and from various parts of the coding or flanking sequences of a gene to optimise the level of anti-sense inhibition. It may be advantageous to include the initiating methionine ATG codon, and perhaps one or more nucleotides upstream of the initiating codon. A suitable fragment may have about 14-23 nucleotides, e.g. about 15, 16 or 17.

An alternative to anti-sense is to use a copy of all or part of the target gene inserted in sense, that is the same, orientation as the target gene, to achieve reduction in expression of the target gene by co-suppression; Angell & Baulcombe (1997) The EMBO Journal 16, 12:3675-3684; and Voinnet & Baulcombe (1997) Nature 389: pg 553).

Double stranded RNA (dsRNA) has been found to be even more effective in gene silencing than both sense or antisense strands alone (Fire A. et al Nature, Vol 391, (1998)). dsRNA mediated silencing is gene specific and is often termed RNA interference (RNAi). RNA interference is a two-step process. First, dsRNA is cleaved within the cell to yield short interfering RNAs (siRNAs) of about 21-23 nt length with 5′ terminal phosphate and 3′ short overhangs (˜2 nt). The siRNAs target the corresponding mRNA sequence specifically for destruction (Zamore P. D. Nature Structural Biology, 8, 9, 746-750, (2001)

RNAi may be also be efficiently induced using chemically synthesized siRNA duplexes of the same structure with 3′-overhang ends (Zamore P D et al Cell, 101, 25-33, (2000)). Synthetic siRNA duplexes have been shown to specifically suppress expression of endogenous and heterologeous genes in a wide range of mammalian cell lines (Elbashir S M. et al. Nature, 411, 494-498, (2001)). See also Fire (1999) Trends Genet. 15: 358-363, Sharp (2001) Genes Dev. 15: 485-490, Hammond et al. (2001) Nature Rev. Genes 2: 1110-1119 and Tuschl (2001) Chem. Biochem. 2: 239-245.

Another possibility is that nucleic acid is used which on transcription produces a ribozyme, able to cut nucleic acid at a specific site—thus also useful in influencing gene expression. Background references for ribozymes include Kashani-Sabet and Scanlon, 1995, Cancer Gene Therapy, 2(3): 213-223, and Mercola and Cohen, 1995, Cancer Gene Therapy, 2(1), 47-59.

A person skilled in the art may produce nucleic acids suitable for use in methods of inhibiting the expression of an Aurora kinase, such as Aurora A, Aurora B or Aurora C, as described above using routine techniques. Oligonucleotides, for example, may be synthesised by phosphotriester or phosphodiester synthesis methods that are well known in the art.

The invention also encompasses a method of treating cancer in an individual comprising administering an Aurora kinase inhibitor and a mitotic spindle assembly inhibitor to said individual.

The administration of therapeutic compositions to an individual is described below.

A cancer suitable for treatment may be resistant or unresponsive to inhibitors of mitotic spindle assembly. For example, Aurora kinase may be over-expressed in the cancer cells relative to non-cancer cells. Over-expression may result from amplification of one or more Aurora kinase genes.

Other related aspects of the invention provide the use of an Aurora kinase inhibitor in the manufacture of a medicament for use in a method of sensitising a tumour cell in an individual to an agent which inhibits mitotic spindle assembly and a method of sensitising a tumour cell in an individual to a mitotic spindle assembly inhibitor comprising administering an Aurora kinase inhibitor.

The tumour cell may over-express an Aurora kinase such as Aurora A, and may be resistant to the mitotic spindle assembly inhibitor in the absence of the Aurora kinase inhibitor.

An Aurora kinase inhibitor may be administered to an individual simultaneously or sequentially with a mitotic spindle assembly inhibitor to sensitise the tumour cell to the mitotic spindle assembly inhibitor (i.e. to reduce the resistance of the cell to the agent).

Another aspect of the invention provides a pharmaceutical composition for use in the treatment of cancer comprising an aurora kinase inhibitor and mitotic spindle assembly inhibitor.

Preferably the pharmaceutical composition further comprises one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

The term “pharmaceutically acceptable” relates to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The invention also encompasses methods of making a pharmaceutical composition comprising admixing an Aurora kinase inhibitor and a mitotic spindle assembly inhibiting agent, as defined above, with one or more pharmaceutically acceptable carriers, excipients, buffers, adjuvants, stabilisers, or other materials, as described herein.

A pharmaceutically acceptable excipient, vehicle or carrier, should be non-toxic and should not interfere with the efficacy of the active ingredient. The precise nature of the carrier or other material will depend on the route of administration, which may be oral, or by injection, e.g. cutaneous, subcutaneous or intravenous Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Suitable carriers, excipients, etc. can be found in standard pharmaceutical texts, for example, Remington's Pharmaceutical. Sciences, 18th edition, Mack Publishing Company, Easton, Pa., 1990.

Formulations and administration regimes which are suitable for use with mitotic spindle assembly inhibitors, in particular taxanes such as paclitaxel, are well known in the art.

Administration is preferably in a “therapeutically effective amount”, this being sufficient to show benefit to the individual. The actual amount administered, and rate and time-course of administration, will depend on the nature and severity of what is being treated. Prescription of treatment, e.g. decisions on dosage etc, is within the responsibility of medical practitioners.

A composition may be administered alone or in combination with other treatments, either simultaneously or sequentially dependent upon the condition to be treated

Other aspects of the invention relate to the evaluation or prognosis of cancer treatment with a mitotic spindle assembly-inhibiting agent. The amount or level of Aurora kinase expression in a tumour cell may be measured in order to predict the responsiveness of a tumour to a mitotic spindle assembly inhibitor, the responsiveness of the cell being reduced when Aurora kinase expression is elevated.

A method of assessing the sensitivity of a tumour cell to a mitotic spindle assembly inhibitor may comprise; determining the expression of an Aurora kinase in said cell.

The tumour cell may be comprised within a sample, for example a tumour biopsy, obtained from an individual. Techniques for obtaining tissue samples for analysis are well known in the art.

The sensitivity or responsiveness of the cell may be determined from the amount of Aurora kinase expression in the cell. When the level of Aurora kinase expression in the cell is increased, the sensitivity of the cell to mitotic spindle assembly inhibiting agents will be decreased (i.e. resistance increases)

An Aurora kinase may include Aurora A, Aurora B or Aurora C.

Aurora kinase expression may be measured by determining the amount of Aurora kinase RNA in said cell. This may be determined by any one of a range of standard techniques such as Northern blotting, RT-PCR or real-time PCR, for example using the Taqman system (Applied Biosystems). These and other suitable techniques are described in Sambrook et al (2001) Cloning: A laboratory Manual 3^{rd} CSH Press NY. Aurora kinase expression may also be determined using RNA expression profiling methods such as DNA microarray hybridisation described in Bowtell & Sambrook (2003) DNA Microarrays: A molecular cloning manual. CSH Press NY.

In some embodiments, Aurora kinase expression may be measured by determining the amplification of the Aurora kinase gene within the cell i.e. the number of copies of the gene within the genome of the cell. This may be determined by any one of a range of standard techniques, such as Southern blotting, PCR, comparative genomic hybridisation (CGH) using microarrays (see for example Bowtell & Sambrook (2003) DNA Microarrays: A molecular cloning manual. CSH Press NY), or in-situ hybridisation methods, which are well known in the art and include fluorescent in-situ hybridisation (FISH). Suitable techniques are described in Sambrook et al (2001) Cloning: A laboratory Manual 3^{rd} Edition CSH NY).

In other embodiments, Aurora kinase expression in said cell may be measured by determining the amount of Aurora polypeptide in said cell.

The amount of Aurora polypeptide in a cell or sample of cells may determined by contacting the cell(s) with an antibody molecule specific for an Aurora kinase (i.e. one or more of Aurora A, B or C). Suitable antibodies may be produced using standard techniques as described above.

A cell, for example from a sample obtained from an individual, may be contacted with an antibody molecule under appropriate conditions for specific binding, and binding determined. The amount of binding is indicative of the amount of Aurora kinase polypeptide in the cell or sample.

Binding of antibody molecules may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule. The mode of determining binding is not a feature of the present invention and those skilled in the art are able to choose a suitable mode according to their preference and general knowledge, Suitable approaches include immunohistochemical staining, Western Blotting, Immunofluorescence, enzyme linked immunosorbent assays (ELISA), radioimmunoassays (RIA), immunoradiometric assays (IRMA) and immunoenzymatic assays (IEMA), including sandwich assays using monoclonal and/or polyclonal antibodies. All of these approaches are well known in the art.

Another aspect of the invention provides a kit for use in a method of assessing the sensitivity of a tumour cell to a mitotic spindle assembly inhibitor.

A kit may comprise amplification primers suitable for amplifying an Aurora kinase nucleic acid sequence, for example an Aurora A, B or C genomic DNA, cDNA or RNA sequence. An oligonucleotide primer for use in nucleic acid amplification may be about 30 or fewer nucleotides in length (e.g. 18, 21 or 24). Generally specific primers are upwards of 14 nucleotides in length, but need not be more than 18-20. Those skilled in the art are well versed in the design of primers for use in processes such as PCR. A kit may further comprise reagents for the detection and quantification of Aurora kinase amplification products.

A kit may comprise a nucleic acid probe which specifically binds to an Aurora kinase sequence. A probe may comprise at least 30, at least 40 or at least 50 contiguous nucleotides of an Aurora kinase sequence described herein, or its complement. The probe may be labelled with a label for detection, for example a fluorescent, enzymatic or radio-label. Other suitable labels may include specific binding members such as biotin. A probe may be suitable for hybridisation with an amplified Aurora kinase nucleic acid sequence, such as a PCR product or a non-amplified Aurora kinase nucleic acid sequence, such as genomic DNA, cDNA or RNA. A kit may further comprise reagents for the detection of the binding of the probe to the Aurora kinase sequence, for example reagents for detecting the presence of the labelled probe.

Oligonucleotide primers and probes may be synthesized using any of a range of techniques well-known in the art, including phosphotriester and phosphodiester synthesis methods.

Hybridisation with Aurora kinase specific oligonucleotides may be conveniently carried out using oligonucleotide arrays, preferably microarrays, to determine the overexpression of Aurora kinase in a cell.

Such microarrays allow miniaturisation of assays, e.g. making use of binding agents (such as nucleic acid sequences) immobilised in small, discrete locations (microspots) and/or as arrays on solid supports or on diagnostic chips. These approaches can be particularly valuable as they can provide great sensitivity (particularly through the use of fluorescent labelled reagents), require only very small amounts of biological sample from individuals being tested and allow a variety of separate assays to be carried out simultaneously. This latter advantage can be useful as it provides an assay for a number of different genes to be carried out using a single sample. Examples of techniques enabling this miniaturised technology are provided in WO84/01031, WO88/1058, WO89/01157, WO93/8472, WO95/18376/WO95/18377, WO95/24649 and EP-A-0373203.

A DNA microarray may be generated using oligonucleotides that have been selected to hybridise with an Aurora kinase nucleic acid sequence. These oligonucleotides may be applied by a robot onto a predetermined location of a glass slide, e.g. at predetermined X,Y cartesian coordinates, and immobilised. Target nucleic acid, for example genomic Aurora kinase DNA or an amplified Aurora kinase sequence, which may be fluorescently labelled RNA or DNA, is introduced on to the DNA microarray and a hybridisation reaction conducted so that sample RNA or DNA binds to complementary sequences of oligonucleotides in a sequence-specific manner. Unbound material is washed away. Gene targets can thus be detected by their ability to bind to complementary oligonucleotides on the array and produce a signal. The fluorescence at each coordinate can be read using a suitable automated detector in order to correlate each fluorescence signal with a particular oligonucleotide. The strength of the signal is indicative of the amount of target nucleic acid present and is thus indicative of the amount of over expression in a cell. Suitable labels that are non-fluorescent are also known in the art.

A kit may comprise an antibody molecule which binds specifically to an Aurora kinase polypeptide. A kit may further comprise reagents for the detection of the binding of the antibody molecule to the Aurora kinase, for example an anti-idiotypic antibody labelled with a reporter molecule.

The components of the kit may be stored in suitable containers such as vials in which the contents are protected from the external environment. The kit may include instructions for use, e.g. in a method of determining the sensitivity of a tumour cell to a mitotic spindle assembly inhibitor, as described herein. A kit wherein the nucleic acid is intended for use in PCR or RT PCR may include one or more other reagents required for the reaction, such as polymerase, nucleosides, buffer solution etc. A kit for use in determining the amount of Aurora kinase expression in a cell may include one or more articles and/or reagents for performance of the method, such as means for providing the test sample itself, e.g. a swab for removing cells from the buccal cavity or a syringe for removing a blood sample (such components generally being sterile).

Various further aspects and embodiments of the present invention will be apparent to those skilled in the art in view of the present disclosure. All documents mentioned in this specification are incorporated herein by reference in their entirety.

Certain aspects and embodiments of the invention will now be illustrated by way of example and with reference to the figures described below.

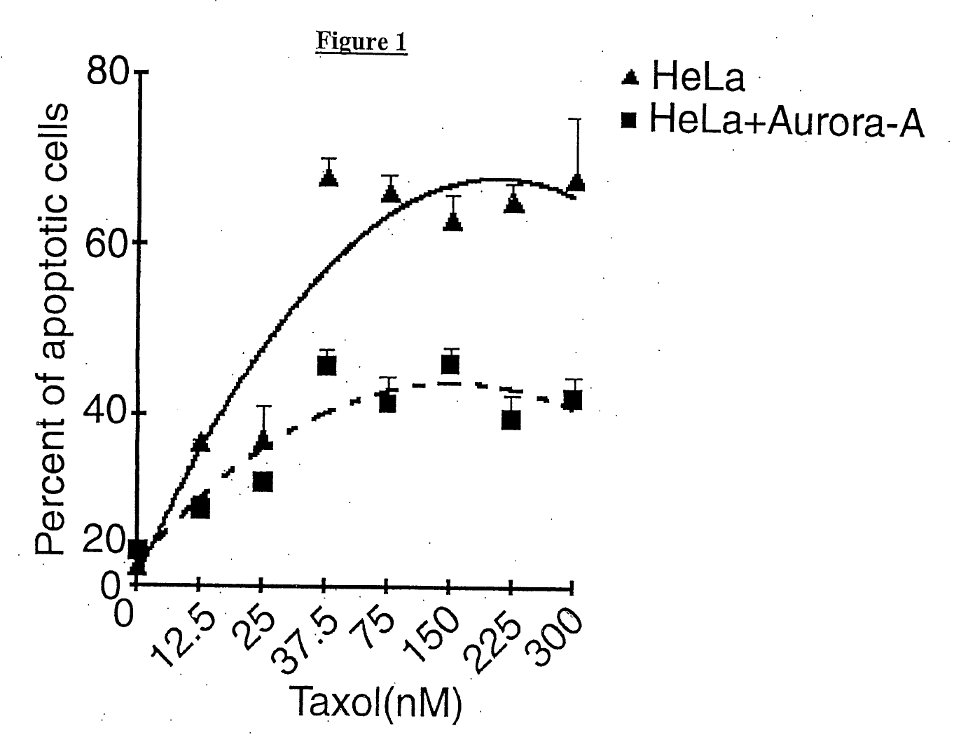

FIG. 1 shows the induction of Taxol resistance by Aurora-A over-expression. The percentage of apoptotic cells enumerated by flow cytometry as described is plotted on the Y-axis, against the dose of Taxol, on the X. The graph compares mock-transfected HeLa cells (closed triangle) against HeLa cells transfected with Aurora-A (closed square). Curves best fitted to the data points by a polynomial equation are shown. Bars depict standard errors from the mean at each data point. Results are typical of two independent repeats.

### EXPERIMENTAL

Materials and Methods

Bi-Cistronic Retroviral Constructs

A cDNA encoding Aurora-A was isolated by RT-PCR using oligonucleotide primers that incorporate appropriate restriction sites as well as a 3-FLAG epitope tag, and cloned between the MluI and BamHI sites of the vector.

Primers used were:

BTAKDO, 5_-GTA CAC GCG TAC CAT GGA CCG ATC TAA AGA
AAA CTG-3;
BTAKFLUP, 5_-CTA GCT CGA GGA TCC TAC TTG TCA TCG
TCG TCC TTG TAG TCT GCC CCA GAC TGT TTG CTA GCT
GAT TC-3.

The empty vector control encodes E-GFP alone. Site-directed mutagenesis to create the Lys162Met (kinase-dead) mutant of Aurora-A was performed with the Quickchange XL kit (Stratagene) using the primers:

A2K162M-1, 5-ATT CTG GCT CTT ATG GTG TTA TTC AAA
GCT CAG CTG-3.
A2K162M-2, 5-GGC TTT CTC CAG CTG AGC TTT GAA TAA
CAC CAT AAG-3.

All constructs were sequenced to verify their authenticity. The previously described cDNA encoding a truncated form of Bub1 with dominant-negative monoactivity (Lee et al., 1999) was subcloned into the pBABEpuro retroviral vector enable selection of transduced cells with puromycin.

Transfection in HeLa

2×10⁵ cells in complete growth medium were plated in a 35 mm dish before transfection using the Gene Juice transfection reagent (Novagen, United Kingdom) according to the manufacturer's protocol. GFP-expressing cells were flow sorted as before, 48 hr after transfection.

Taxol Sensitivity Assay

Equal numbers of flow sorted mock-transfected or Aurora-A-transfected HeLa cell were plated on 6 well plates in DMEM (Life Technologies, Grand Island, N.Y.) containing 10% FBS. Taxol (Sigma Chemical Co.) at different concentrations was added fresh from a stock prepared in DMSO. The cells were exposed to the drug for 72 hr after which they were analyzed by propidium iodide staining and flow cytometry as described earlier.

Cell Cycle Analysis

Cells collected at the indicated time points were centrifuged at 1000 rpm for 5 min at room temperature. 1×10⁶ cells were resuspended in 0.5 ml PBS, fixed by adding 4.5 ml of ice-cold 70% ethanol, and incubated overnight in fixative at 4° C. Before analysis, fixed cells were washed in PBS and incubated with propidium iodide staining solution (0.1% Triton.X-100, PBS; 0.02 mg/ml propidium iodide; 0.2 mg/ml RnaseA) for 30 min at room temperature. Analysis was on a FACSCalibur cytometer (Becton-Dickinson, California) using CellQuest software, with appropriate gating on the FL2-A and FL2-W channels to exclude cell aggregates. Ten thousand events were analyzed per sample.

Results

AURORA-A Overexpression and Paclitaxel Sensitivity

HeLa cells are known to be highly sensitive to Taxol-induced apoptosis. Mock transfected and Aurora-A-overexpressing HeLa cells were treated with different concentrations of Taxol for 72 hr. The percentage of apoptotic cells was then measured using propidium iodide staining and flow cytometry to enumerate the sub G1 (<2N DNA content) population.

Aurora-A overexpression was found to induce a striking increase in resistance to paclitaxel-induced apoptosis (FIG. 1).

Amplification of the 20q13.2 region containing the AURORA-A gene occurs in a high proportion of epithelial cancers, with estimates ranging from 20%-60% for breast and colorectal tumors. Amplification is accompanied by elevated Aurora-A expression. The levels of Aurora-A over-expression achieved in the experiments herein correspond to those observed for many human cancers and provide indication that the cancer-associated amplification of Aurora kinase genes may give rise to the biological effects which were observed in the experiments described herein. Agents such as paclitaxel (taxol™), which are used widely in cancer chemotherapy, arrest cell division by perturbing mitotic spindle assembly, a process that is monitored by the spindle assembly checkpoint. Aurora over-expression dysregulates this checkpoint mechanism and is shown herein to confer increased resistance to Taxol in a human epithelial cancer cell line. Inhibition of Aurora activity may therefore be useful in the development of taxane-based therapies for the treatment of cancer.

1. A method of treating cancer in an individual comprising administering an Aurora kinase inhibitor and a mitotic spindle assembly inhibitor to said individual.

2. A method according to claim 1 wherein said tumour cell is resistant to said agent that inhibits mitotic spindle assembly.

3. A method according to claim 1 wherein the mitotic spindle assembly inhibitor is a taxane.

4. A method according to claim 3 wherein the taxane is paclitaxel or a derivative thereof.

5. A method according to claim 1 wherein the Aurora kinase inhibitor is selected from the group consisting of 4-(4-(N benzoylamino)anilino)-6-methoxy-7-(3-(1 morpholino)propoxy)quinazoline and Hesperadin.

6. A method according to claim 1 wherein the Aurora kinase inhibitor is an antibody molecule which specifically binds to an Aurora kinase.

7. A method according to claim 1 wherein the Aurora kinase inhibitor is a sense or anti-sense nucleic acid molecule that inhibits the expression of an Aurora kinase.

8. A method according to claim 1 wherein the cancer is an epithelial cancer.

9. A method according to claim 8 wherein the epithelial cancer is skin, thyroid, colon, pancreas, lung, prostate, cervical, ovarian or breast cancer.

10. A method according to claim 1 wherein the cancer is liver, kidney or brain cancer.

11. A method of sensitising a tumour cell in an individual to an agent which inhibits mitotic spindle assembly comprising administering an Aurora kinase inhibitor to the individual.

12. A method according to claim 11 wherein said tumour cell is resistant to said agent which inhibits mitotic spindle assembly.

13. A method according to claim 11 wherein the mitotic spindle assembly inhibitor is a taxane.

14. A method according to claim 13 wherein the taxane is paclitaxel or a derivative thereof.

15. A method according to claim 11 wherein the Aurora kinase inhibitor is selected from the group consisting of 4-(4-(N benzoylamino)anilino)-6-methoxy-7-(3-(1 morpholino)propoxy)quinazoline and Hesperadin.

16. A method according to claim 11 wherein the Aurora kinase inhibitor is an antibody molecule which specifically binds to an Aurora kinase.

17. A method according to claim 11 wherein the Aurora kinase inhibitor is a sense or anti-sense nucleic acid molecule which inhibits the expression of an Aurora kinase.

18. A pharmaceutical composition comprising an Aurora kinase inhibitor and a mitotic spindle assembly inhibitor.

19. A pharmaceutical composition according to claim 18 comprising a pharmaceutically acceptable excipient.

20. A composition according to claim 18 wherein the mitotic spindle assembly inhibitor is a taxane.

21. A composition according to claim 20 wherein the taxane is paclitaxel or a derivative thereof.

22. A composition according to claim 18 wherein the Aurora kinase inhibitor is selected from the group consisting of 4-(4-(N benzoylamino)anilino)-6-methoxy-7-(3-(1 morpholino)propoxy)quinazoline and Hesperadin.

23. A method of determining the resistance of a tumour cell to a mitotic spindle assembly inhibitor comprising; measuring the, expression of Aurora kinase in said tumour cell, elevated expression of Aurora kinase in said tumour cell relative to a non-tumour cell being indicative of the resistance of said tumour cell to the agent.

24. A method according to claim 23 wherein the mitotic spindle assembly inhibitor is a taxane.

25. A method according to claim 24 wherein the taxane is paclitaxel.

26. A method according to claim 23 wherein the expression of Aurora kinase in said cell is measured by determining the amount of Aurora kinase RNA in said cell.

27. A method according to claim 23 wherein the expression of Aurora kinase in said cell is measured by determining the copy number in said cell of nucleic acid that encodes Aurora kinase.

28. A method according to claim 23 wherein the expression of Aurora in said cell is measured by determining the amount of Aurora kinase polypeptide in said cell.

29. A method according to claim 23 wherein the Aurora kinase is Aurora A.

30. A kit for use in a method of determining the sensitivity of a tumour cell to a mitotic spindle assembly inhibitor comprising oligonucleotide primers suitable for specific amplification of an Aurora kinase nucleic acid sequence.

31. A kit for use in a method of determining the sensitivity of a tumour cell to a mitotic spindle assembly inhibitor comprising an antibody molecule which binds specifically to an Aurora kinase.

32. A kit according to claim 31 comprising a labelled secondary antibody and one or more label detection reagents.

33-51. (canceled)
